(19) United States
(12) Patent Application Publication
(10) Pub. No.: US 2026/0155227 A1
(43) Pub. Date: Jun. 4, 2026

(54) INSULIN DYNAMICS OPTIMIZATION

(71) Applicant: GNQ INSILICO, INC., PLEASANTON, CA (US)

(72) Inventors: SUDHIR SAXENA, PLEASANTON, CA (US); SARWAT ANWER, PLEASANTON, CA (US)

(21) Appl. No.: 19/404,832

(22) Filed: Dec. 1, 2025

Related U.S. Application Data
(60) Provisional application No. 63/727,027, filed on Dec. 2, 2024.

(51) Int. Cl. G16H 20/10; G16H 40/60

(52) U.S. Cl. CPC G16H 20/10; G16H 40/60

(57) ABSTRACT

Generating one or more health-related recommendations including processing a plurality of data sources associated with one or more health-related systems to generate integrated pathway parameters; constructing a mathematical representation of a patient state based on the integrated pathway parameters; dynamically refining the mathematical representation of the patient state using a machine-learning process configured to adaptively update parameter weights in response to incoming health-related data; and generating, in response to dynamically refining the mathematical representation of the patient state, one or more predictive outputs corresponding to the one or more health-related recommendations.

### CROSS-REFERENCE TO RELATED APPLICATION

This application claims the priority benefit under 35 U.S.C. § 119 (e) of U.S. Provisional Application No. 63/727,027, filed on Dec. 2, 2024, the disclosure of which is incorporated by reference in its entirety as if fully set forth herein.

### FIELD

The field of the present disclosure relates to medical data processing and adaptive control systems. More specifically, the disclosure relates to systems and methods for adaptive data processing, machine learning, and predictive optimization used in the monitoring and management of physiological processes.

### BACKGROUND

The statements in this section merely provide background information related to the present disclosure and may not constitute prior art.

Insulin management systems utilized within modern clinical and personal health environments regulate glucose levels through continuous glucose monitoring, insulin pumps, and staged therapeutic protocols. Most existing implementations rely on static diagnostic metrics and fixed dosing algorithms that fail to capture the temporal and biological variability of insulin activity. Conventional approaches analyze discrete glucose readings or aggregated HbAlc values without integrating broader physiological, genetic, or behavioral inputs that influence insulin sensitivity and secretion. These systems often adjust therapy reactively after glucose deviations occur, resulting in inefficiencies in glycemic control and limited personalization of treatment. As patient data streams expand to include multi-omics analyses, wearable sensor inputs, and electronic medical records, conventional systems lack mechanisms to synthesize information derived from such patient data streams into an adaptive, patient-specific framework.

Current computational frameworks for insulin regulation do not fully capture the complexity of human metabolism or the dynamic interplay between physiological, genetic, and environmental factors that influence insulin function. Many implementations rely on linear models or fixed algorithms that cannot adapt to evolving biological conditions or variations across patient populations. Data from multi-omics sources, continuous glucose monitors, and clinical records often remain siloed, limiting the ability to derive predictive insights across molecular and systemic levels. As a result, existing systems provide limited accuracy in forecasting insulin needs, respond slowly to physiological fluctuations, and lack the scalability to support individualized or population-level optimization. The present disclosure addresses these and other issues related to providing a means for adaptive and data-driven management of physiological processes through continuous analysis, modeling, and optimization of patient-specific biological states.

### SUMMARY

According to embodiments of the present disclosure, various systems, methods, and computer program products for managing insulin dynamics are described herein. In various aspects, a computer-implemented method for generating one or more health-recommendations associated with the management of insulin dynamics includes processing, by a computing device, a plurality of data sources associated with one or more health-related systems to generate integrated pathway parameters; constructing, by the computing device, a mathematical representation of a patient state based on the integrated pathway parameters; dynamically refining, by the computing device, the mathematical representation of the patient state using a machine-learning process configured to adaptively update parameter weights in response to incoming health-related data; and generating, by the computing device and in response to dynamically refining the mathematical representation of the patient state, one or more predictive outputs corresponding to one or more health-related recommendations. In various aspects, a system may include a memory and one or more processing devices, operatively coupled to the memory, where the one or more processing devices performs similar steps to those described above. In various aspects, a computer program product may include a non-transitory computer-readable medium storing processor-executable instructions that, when executed, carry out the computer-implemented methods described above.

Although embodiments are described in the context of insulin regulation, the described computational framework can be applied to any system requiring adaptive prediction and optimization, including other physiological systems, therapeutic modeling processes, or non-medical dynamic control applications.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

In order that the disclosure may be well understood, there will now be described various forms thereof, given by way of example, reference being made to the accompanying drawings, in which:

FIG. 1 shows a block diagram of an example system configured for managing insulin dynamics according to embodiments of the present disclosure;

FIG. 2 illustrates a diagram of an example computer system configured to manage insulin dynamics according to embodiments of the present disclosure;

FIG. 3 shows a flowchart of an example method for generating health-related recommendations associated with the management of insulin dynamics according to embodiments of the present disclosure;

FIG. 4 shows a flowchart of another example method for generating health-related recommendations associated with the management of insulin dynamics according to embodiments of the present disclosure;

FIG. 5 shows a flowchart of yet another example method for generating health-related recommendations associated with the management of insulin dynamics according to embodiments of the present disclosure;

FIG. 6 shows a flowchart of a further example method for generating health-related recommendations associated with the management of insulin dynamics according to embodiments of the present disclosure;

FIG. 7 shows a flowchart of an additional example method for generating health-related recommendations associated with the management of insulin dynamics according to embodiments of the present disclosure;

FIG. 8 shows a flowchart of another example method for generating health-related recommendations associated with the management of insulin dynamics according to embodiments of the present disclosure;

FIG. 9 shows a flowchart of yet another example method for generating health-related recommendations associated with the management of insulin dynamics according to embodiments of the present disclosure;

FIG. 10 sets forth a block diagram of a cloud service provider architecture in accordance with some embodiments of the present disclosure; and

FIG. 11 is a block diagram of an electronic device in a network environment according to embodiments of the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

Management of insulin dynamics in clinical and personal health environments remains limited by systems that rely on static data inputs and fixed dosing algorithms. These approaches evaluate glucose and insulin data as discrete measurements, without accounting for the broader biological, genetic, and temporal interactions that govern metabolic function. As a result, current systems lack the ability to predict insulin requirements with precision or to adjust therapy dynamically as physiological conditions change. This limitation is further compounded by fragmented data ecosystems in which multi-omics profiles, clinical records, and real-time sensor data are not integrated within a unified computational framework, reducing the accuracy and adaptability of insulin modeling and therapeutic recommendations.

To address such challenges the present disclosure sets forth various systems and methods for managing insulin dynamics. The described systems integrate multi-source biomedical data, pathway-based modeling, and adaptive computational processes to represent and refine patient-specific physiological states over time. Through this integration, the disclosed technology enables continuous, data-driven adjustment of insulin management strategies that evolve alongside patient conditions. Benefits provided include, but are not limited to, enhanced precision in predicting insulin demand, supply, and efficiency, continuous adaptation to new biological inputs, reduced therapeutic lag, and improved support for automated and personalized treatment.

As used herein, the term “patient state” refers to a quantitative or computational representation of a biological or physiological condition of a patient derived from multi-omics data, clinical records, and real-time health information. A patient state may include variables representing insulin levels, glucose concentrations, metabolic activity, and other biomarkers that collectively define the physiological condition of the patient at a given time. The patient state may be dynamically updated as new data becomes available from external data sources such as wearable sensors, continuous glucose monitoring devices, or electronic medical records. The mathematical representation of a patient state, as described herein, serves as the foundation for predictive modeling, optimization, and therapeutic decision-making.

As used herein, the term “integrated pathway parameters” refers to data-derived variables that describe relationships among genetic, metabolic, and physiological processes within a biological system. Integrated pathway parameters may be generated by processing heterogeneous data from multiple sources (e.g., including multi-omics datasets, clinical databases, and real-time sensor inputs) to identify functional correlations among biological pathways. Integrated pathway parameters may further include variables derived from lifestyle and environmental data, such as exercise patterns, dietary behavior, sleep metrics, stress indicators, activity logs, or environmental exposures (e.g., temperature, humidity, or air quality), whether obtained from user input, companion applications, or third-party data providers. The integrated pathway parameters may capture dynamic interactions between insulin demand, supply, and efficiency, thereby enabling the construction of a mathematical representation of the patient state used for adaptive modeling and prediction.

As used herein, the term “mathematical representation” refers to a computational model or expression that encodes biological, clinical, and temporal relationships associated with a patient state. The mathematical representation may include linear, non-linear, or optimization-based formulations, such as a quadratic unconstrained binary optimization (QUBO) model, that define dependencies among pathway parameters and physiological constraints. In one or more embodiments, the mathematical representation may be expressed as minimizing an objective function of the form Q(x)=x^{T}Px+q^{T}x+c, where P represents an interaction matrix encoding pairwise relationships among pathway variables, q represents linear coefficients derived from integrated pathway parameters, and c represents a constant term. The mathematical representation may be refined iteratively using machine learning or optimization algorithms to improve predictive accuracy and alignment with an observed clinical outcome.

As used herein, the term “predictive output” refers to a generated result derived from the mathematical representation of a patient state, which provides insight into physiological behavior or therapeutic needs. Predictive outputs may include, but are not limited to, insulin-demand and supply forecasts, efficiency metrics, or recommendations for clinical or lifestyle interventions. Predictive outputs may be transmitted to healthcare providers, patient interfaces, or automated devices for real-time management of insulin dynamics.

As used herein, the term “health-related recommendation” refers to a patient-specific output derived from predictive modeling that provides actionable clinical or behavioral guidance. A health-related recommendation may include instructions for insulin dosage adjustment, lifestyle modifications, or alerts indicating anomalous physiological conditions. In some embodiments, health-related recommendations may be automatically applied to a drug-delivery device for closed-loop control of insulin administration, or displayed to a clinician or patient through a user interface for manual review and implementation.

As used herein, the term “machine-learning process” refers to an adaptive computational procedure that enables a system to refine predictive models or optimize parameters based on new data. The machine-learning process may employ one or more algorithms, including graph neural networks, reinforcement learning, neural-network prediction models, or genetic algorithms, to iteratively adjust variable weights, improve model convergence, and enhance predictive performance. The machine-learning process described herein operates in conjunction with optimization and validation steps to enable continuous, data-driven management of insulin dynamics.

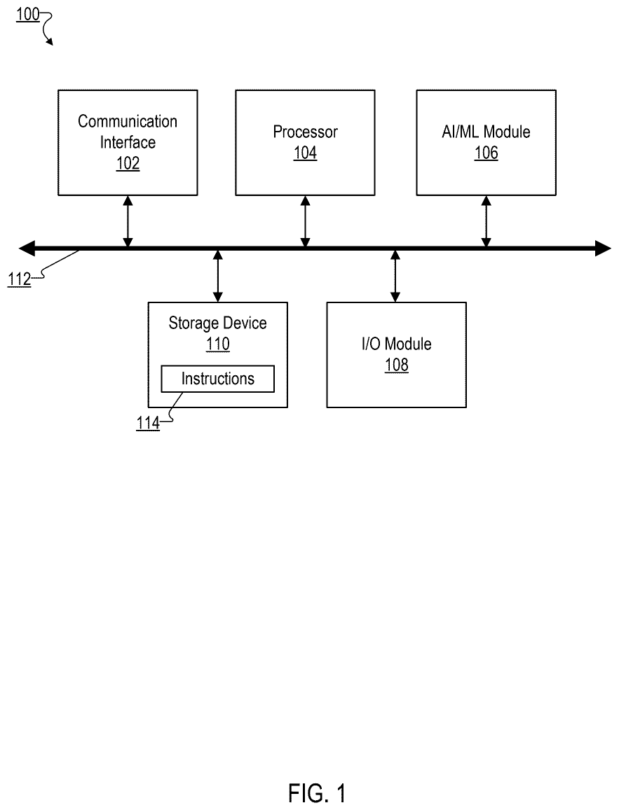

Example methods, systems, and products for managing insulin dynamics in accordance with embodiments of the present disclosure are described with reference to the accompanying drawings, beginning with FIG. 1. In one or more embodiments, FIG. 1 illustrates an example computing system 100 that may be specifically configured to perform one or more of the processes described herein associated with the management of insulin dynamics. As shown in FIG. 1, the computing system 100 may include a communication interface 102, a processor 104, an artificial intelligence and machine learning (AI/ML) module 106, an input/output (I/O) module 108, and a storage device 110 that stores computer-executable instructions 114. The communication interface 102, the processor 104, the AI/ML module 106, the I/O module 108, and the storage device 110 are communicatively connected one to another via a communication infrastructure 112. The computer-executable instructions 114, when executed by the processor 104, may cause the computing system 100 to perform operations for managing insulin dynamics, including processing data from one or more health-related systems, constructing and refining mathematical representations of patient states, and generating predictive outputs associated with health-related recommendations. While an exemplary computing system 100 is shown in FIG. 1, the components illustrated are not intended to be limiting, and additional or alternative components may be used in other embodiments. Components of the computing system 100 shown in FIG. 1 will now be described in additional detail.

The communication interface 102 may be configured to communicate with one or more external systems that provide health-related input data to, or receive generated output data from, the computing system 100. For example, the communication interface 102 may enable access to multi-omics datasets, clinical records, continuous glucose monitoring data, and other patient-specific repositories, as well as interfaces that distribute predictive outputs or health-related recommendations to client devices, clinical dashboards, or automated drug-delivery systems. Examples of the communication interface 102 include, without limitation, a wired or wireless network interface, a high-speed interconnect, an application programming interface (API) gateway, or another communication module configured for data exchange between computing nodes in a distributed or cloud-based computational environment. In some embodiments, the communication interface 102 may include encryption, authentication, or other security mechanisms to ensure secure transmission of sensitive medical and biological data across clinical and networked computing environments.

The processor 104 generally represents one or more processing units capable of executing the operations of the computing system 100 associated with the management of insulin dynamics. The processor 104 may execute the computer-executable instructions 114 stored in the storage device 110 to perform operations such as processing multi-source patient data, constructing mathematical representations of patient states, dynamically refining such representations using machine-learning algorithms, and generating predictive outputs corresponding to health-related recommendations. The processor 104 may include one or more general-purpose processing units, graphics processing units (GPUs), tensor processing units (TPUs), or other AI-optimized accelerators configured to perform large-scale matrix computations, optimization routines, and adaptive learning operations. In some embodiments, the processor 104 may coordinate distributed processing across multiple computing nodes or cloud-based instances to enable real-time model updates, continuous data ingestion, and low-latency prediction for adaptive management of insulin dynamics.

The AI/ML module 106 may be configured to perform adaptive modeling and analysis operations that support management of insulin dynamics. The AI/ML module 106 may receive data processed by the processor 104, including integrated pathway parameters derived from multi-omics sources, clinical records, and real-time sensor inputs. Using the processed data, the AI/ML module 106 may construct mathematical representations of patient states and iteratively refine the mathematical representations based on feedback from newly acquired health-related information. In one or more embodiments, the AI/ML module 106 may employ graph neural networks, reinforcement learning algorithms, neural network prediction models, or genetic algorithm optimization routines to capture and adapt complex physiological relationships. When operating in coordination with the processor 104, the AI/ML module 106 may continuously update weighting parameters, evaluate constraint boundaries, and generate intermediate computational outputs used to produce predictive health-related recommendations. Through these operations, the AI/ML module 106 enables the computing system 100 to perform real-time, data-driven modeling that enhances predictive accuracy, improves adaptation to individual patient variability, and supports safe and efficient management of insulin dynamics.

The I/O module 108 may include one or more input and output devices configured to receive user input and present output data generated by the computing system 100. The I/O module 108 may include any suitable combination of hardware, firmware, and software that supports data entry, configuration, and visualization capabilities. For input, the I/O module 108 may include interfaces for uploading patient datasets, adjusting operational parameters such as model update frequency or constraint thresholds, and selecting processing modes related to data integration, modeling, or predictive output generation. Input devices may include keyboards, touchscreens, configuration panels, or web-based control dashboards accessible to clinicians or system administrators. For output, the I/O module 108 may include displays, dashboards, or graphical user interfaces (GUIs) configured to present system-generated outputs such as insulin-demand and supply predictions, therapy recommendations, model performance metrics, or data integrity indicators. The I/O module 108 may further provide visualization tools that enable monitoring of the mathematical representation of patient states in real-time, including graphical trends of insulin efficiency, metabolic variability, or confidence intervals associated with predictive outputs. In some embodiments, the I/O module 108 may also include external interfaces for exporting processed results or control signals to downstream clinical systems, such as APIs, connected medical devices, or automated drug-delivery platforms.

The storage device 110 may include one or more types of non-volatile storage media and may be configured to store the computer-executable instructions 114 along with datasets, model parameters, and artifacts generated or used by the computing system 100. The storage device 110 may maintain patient-specific data such as multi-omics profiles, clinical records, and time-series sensor readings, as well as intermediate computational results including integrated pathway parameters, model weight matrices, and validation thresholds. In some embodiments, the storage device 110 may include structured databases or repositories that organize and index historical patient states, predictive outputs, and refinement history for efficient retrieval and model reinitialization. The storage device 110 may further maintain persistent logs of system activity, including model performance metrics, constraint validation results, and data-ingestion events, to support traceability, compliance, and iterative model improvement. In certain embodiments, the storage device 110 may integrate with secure cloud-based repositories to facilitate distributed access, redundant storage, and scalable management of patient-state information across clinical or research environments.

The communication infrastructure 112 represents the internal interconnect architecture that links the communication interface 102, the processor 104, the AI/ML module 106, the I/O module 108, and the storage device 110 within the computing system 100. The communication infrastructure 112 ensures efficient data exchange of integrated pathway parameters, model representations, intermediate computational results, and health-related recommendations between components of the computing system 100. The communication infrastructure 112 may be implemented using one or more buses, fabrics, or network topologies optimized for high-throughput, low-latency data transfer across computational resources, enabling real-time synchronization of data and model updates required for continuous management of insulin dynamics.

The computer-executable instructions 114 stored in the storage device 110 may define operations that, when executed by the processor 104, enable the computing system 100 to perform one or more processes associated with the management of insulin dynamics. These instructions 114 may include routines for data acquisition, model construction, adaptive refinement, validation, and output generation. In some embodiments, the computer-executable instructions 114 may further include algorithms for processing and normalizing biomedical data, constructing mathematical representations of patient states, and executing optimization techniques such as quadratic unconstrained binary optimization. The instructions 114 may also define machine-learning workflows that dynamically update model parameters based on real-time input data and safety or efficacy constraints. When executed, the computer-executable instructions 114 coordinate the functions of the communication interface 102, the processor 104, the AI/ML module 106, the I/O module 108, and the storage device 110, thereby enabling the computing system 100 to operate as an integrated platform for continuous, adaptive management of insulin dynamics.

By combining these components, the computing system 100 is specifically configured to execute the processes described herein for managing insulin dynamics. In particular, the computing system 100 supports operations at least for processing multi-source health data, constructing and refining mathematical representations of patient states, validating those representations against physiological constraints, and generating predictive outputs that inform therapeutic recommendations or automated control actions. The integrated architecture of the computing system 100 enables high-throughput data ingestion, continuous model adaptation, and secure communication across clinical and distributed computing environments. Through coordinated operation of its hardware and software components, the computing system 100 provides a scalable and adaptive platform capable of supporting real-time, patient-specific insulin management in accordance with embodiments of the present disclosure.

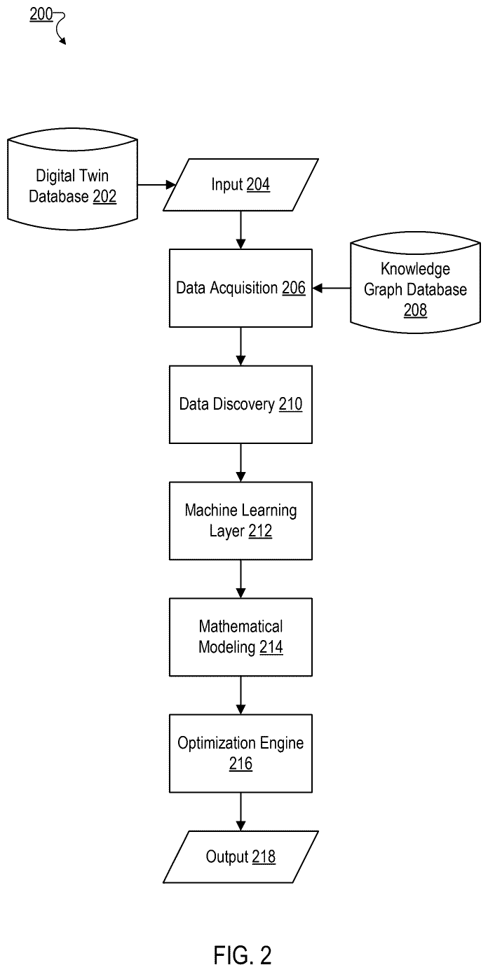

FIG. 1 therefore illustrates an example computing system 100 configured to perform operations associated with the management of insulin dynamics through data integration, adaptive modeling, and real-time prediction. The described configuration enables the computing system 100 to acquire and process health-related data from multiple external sources, refine mathematical representations of patient states using machine-learning techniques, and generate validated outputs suitable for clinical interpretation or automated therapeutic response. The modular design of the computing system 100 allows deployment within local clinical infrastructures, cloud-based environments, or hybrid architectures, supporting scalability across individual and population-level use cases. The computing system 100 thus serves as a foundational architecture for executing the systems and methods described in the subsequent figures. FIG. 2 illustrates an example system architecture that builds upon the computing system 100 and further defines the functional layers and data flow for implementing continuous management of insulin dynamics in accordance with embodiments of the present disclosure.

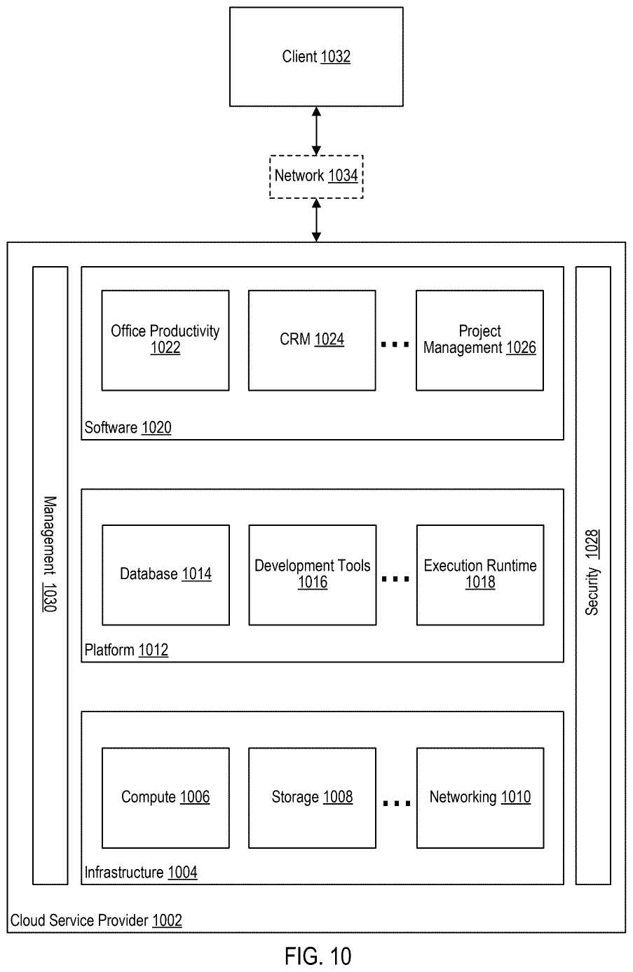

For further explanation, FIG. 2 sets forth a block diagram of an example system 200 configured for managing insulin dynamics in accordance with embodiments of the present disclosure. In one or more examples, the example system 200 can be representative of the computing system 100 illustrated in FIG. 1. The system 200 may be implemented using one or more instances of the computing system 100 described with reference to FIG. 1 or within other suitable computing environments as would be understood by one skilled in the art, such as those described with reference to FIGS. 10 and/or 11. In some embodiments, the components of the system 200 may be implemented within a single computing environment that performs data processing, modeling, and optimization in an integrated manner. In other embodiments, the components of the system 200 may be distributed across multiple computing devices or networked environments configured to exchange data and model parameters in real-time. The components of the system 200 may be implemented using one or more software applications, specialized hardware accelerators, or combinations thereof configured to perform high-efficiency data integration, adaptive machine learning, and continuous optimization operations associated with the management of insulin dynamics. In one or more embodiments, the system 200 may operate within a continuous feedback loop in which newly acquired data, refined model outputs, and updated mathematical representations of the patient state are repeatedly cycled through various operations to maintain a real-time and adaptive depiction of insulin dynamics, as is described herein.

In one or more embodiments, the system 200 may be deployed across multiple healthcare and research contexts, including drug development, clinical trial execution, clinical management, and personal health monitoring. In drug development and clinical trial settings, the system 200 may support therapy-response simulation, cohort stratification, dose optimization, and adaptive protocol design through use of digital twins representing diverse patient populations. In clinical management environments, the system 200 may provide point-of-care decision support and personalized therapeutic guidance, while in personal monitoring contexts, the system 200 may operate on data provided by wearable devices, companion applications, or user input to support individualized wellness and metabolic regulation.

The system 200 of FIG. 2 includes an input 204, a data acquisition component 206, a data discovery component 210, a machine learning layer 212, a mathematical modeling component 214, an optimization engine 216, and an output 218. Together, these components define a layered computational framework configured to receive, process, and transform biomedical data into adaptive patient-state models to be utilized in the management of insulin dynamics. The input 204 can include information from a digital twin database 202 that stores multi-omics profiles, clinical records, and real-time streaming health data associated with one or more patients, as well as lifestyle and environmental information such as dietary intake, activity levels, exercise type or duration, sleep patterns, stress indicators, or ambient conditions (e.g., temperature, humidity, or air quality). Such lifestyle and environmental data may be obtained from direct user input, connected applications, or third-party data services in addition to, or instead of, sensor-derived sources.

The data acquisition component 206 aggregates and preprocesses this information together with biological pathway data that can originate from a knowledge graph database 208.

The data discovery component 210 identifies relationships among biological and clinical parameters and generates integrated pathway parameters that capture the interconnected nature of insulin regulation. The machine learning layer 212 applies adaptive algorithms to integrate and continuously update the integrated pathway parameters as new patient data becomes available. The mathematical modeling component 214 constructs a mathematical representation of the patient state using the integrated pathway parameters, and the optimization engine 216 refines the mathematical representation through iterative computation to improve accuracy and adherence to physiological and safety constraints. In one or more embodiments, the refinement provided by the optimization engine 216 can generate an output 218 that can include one or more predictive outputs corresponding to health-related recommendations, thereby enabling continuous, data-driven management of insulin dynamics.

More specifically, the input 204 can include information from the digital twin database 202, which stores comprehensive, patient-specific datasets used to support modeling and optimization within the system 200. The digital twin database 202 can include multi-omics profiles, clinical records, and real-time streaming health data collected from continuous glucose monitoring devices, wearable sensors, and electronic medical record systems, and may further store synthesized or simulated digital twins generated from aggregated population-level data, modeled physiological relationships, or behavioral patterns derived from a machine-learning source. This information can provide a multidimensional view of the physiological state of each patient, forming the foundation for individualized modeling and prediction of insulin behavior. The input 204 can serve as a transfer point through which data originating from the digital twin database 202 is provided to the data acquisition component 206 for preprocessing, normalization, and integration with additional biological pathway data received from the knowledge graph database 208. In one or more embodiments, the input 204 can also facilitate access to updates from external or distributed databases to ensure that the data utilized by the system 200 reflects the most current and complete representation of patient-specific biological information.

The data acquisition component 206 may be configured to receive and aggregate information received as the input 204 along with biological pathway data originated from the knowledge graph database 208. The knowledge graph database 208 can include structured biomedical information describing genetic, metabolic, and signaling pathways, molecular interactions, as well as clinically validated relationships associated with the regulation of insulin. The data acquisition component 206 can preprocess and align the information provided as the input 204, which can include data received from the digital twin database 202, to ensure consistency, accuracy, and temporal synchronization across diverse data types. In one or more embodiments, the data acquisition component 206 can perform data cleaning, normalization, interpolation, and transformation operations to reconcile variations in format, scale, or timing among the multiple data sources. The processed and aligned data can then be merged into a unified dataset suitable for analysis and interpretation by the data discovery component 210. In certain embodiments, the data acquisition component 206 can further perform quality control operations, including outlier detection and completeness checks, to maintain the reliability of the aggregated data for downstream modeling.

The data discovery component 210 may be configured to analyze the aggregated and preprocessed data received from the data acquisition component 206 to identify relevant biomarkers, pathway interactions, and contextual relationships that influence insulin regulation, including lifestyle-driven and environmental influences such as dietary behavior, physical activity patterns, sleep quality, stress levels, or exposure to environmental conditions. The data discovery component 210 can apply analytical techniques, including statistical correlation, pathway mapping, and clustering analysis, to reveal interdependencies among genetic, metabolic, and physiological parameters. These analytical operations can generate integrated pathway parameters that capture both direct and indirect influences within a biological system of a patient. In one or more embodiments, the data discovery component 210 can incorporate biological pathway structures derived from the knowledge graph database 208 to enhance the interpretability and precision of the identified relationships. The integrated pathway parameters produced by the data discovery component 210 can serve as inputs to the machine learning layer 212, which can utilize integrated pathway parameters to model dynamic insulin-related behaviors and continuously refine predictions based on evolving patient data.

The machine learning layer 212 may be configured to apply adaptive algorithms to integrate and continuously update the integrated pathway parameters generated by the data discovery component 210. The machine learning layer 212 can include one or more models such as graph neural networks, reinforcement learning models, neural-network prediction models, or genetic-algorithm optimization routines designed to represent nonlinear and dynamic relationships among biological, clinical, and environmental variables influencing insulin dynamics. In one or more embodiments, the machine learning layer 212 may be trained, calibrated, or periodically retrained using real-world data stored in the digital twin database 202, including historical multi-omics data, longitudinal clinical records, sensor-derived information, lifestyle and environmental inputs, and population-level outcome data, enabling the machine-learning process to learn biological, temporal, and behavioral patterns that cannot be inferred from patient-specific data alone.

The machine learning layer 212 can perform feature weighting, dependency mapping, and temporal pattern recognition to quantify and track the impact of physiological and behavioral factors on insulin demand, supply, and efficiency. In one or more embodiments, the machine learning layer 212 can incorporate real-time data updates received as the input 204 to ensure that model parameters reflect current patient conditions. The outputs of the machine learning layer 212 can include refined feature sets and updated parameter weightings, which can be provided to the mathematical modeling component 214 for constructing a mathematical representation of the patient state.

The mathematical modeling component 214 may be configured to construct a mathematical representation of the patient state using the integrated pathway parameters and refined weightings generated by the machine learning layer 212. The mathematical modeling component 214 can translate the complex relationships among clinical, biological, and physiological variables into a quantitative model that represents dynamic insulin behavior. In one or more embodiments, the mathematical modeling component 214 can formulate a quadratic unconstrained binary optimization (QUBO) model that encodes the relationships among the integrated pathway parameters, enabling representation of interdependent biological and temporal processes within a computational optimization framework. The mathematical representation of the patient state generated by the mathematical modeling component 214 can reflect the interaction between insulin demand, supply, and efficiency over time. The mathematical modeling component 214 can provide the constructed representation to the optimization engine 216 for iterative computation and refinement.

The optimization engine 216 may be configured to refine the mathematical representation of the patient state through iterative computation and validation. The optimization engine 216 can apply one or more computational optimization frameworks to adjust parameter values and evaluate the resulting outputs to ensure accuracy, stability, and clinical reliability. In one or more embodiments, the optimization engine 216 can implement constraint-based and machine-learning-assisted techniques to refine the representation generated by the mathematical modeling component 214. The optimization engine 216 can perform operations such as solution validation, constraint checking, and performance tracking to confirm adherence to predefined physiological and safety parameters. Through these iterative computations, the optimization engine 216 can improve predictive precision, reduce error across temporal predictions, and generate refined data outputs that represent the most current and accurate state of insulin dynamics for a given patient. The refined mathematical representation produced by the optimization engine 216 can then be provided as the output 218 for generation of health-related recommendations.

In one or more examples, the output 218 may include one or more predictive outputs corresponding to health-related recommendations derived from the refined mathematical representation of the patient state. The predictive outputs can include clinical insights, lifestyle recommendations, insulin-demand and supply predictions, or automated control instructions transmitted to therapeutic devices. In one or more embodiments, the output 218 can include information that incorporates safety parameters, confidence intervals, or alerts for atypical patterns to support reliable interpretation and clinical decision-making. The output 218 can provide the included recommendations to user interfaces, clinical dashboards, or automated delivery systems, enabling real-time access to adaptive and patient-specific information. In certain embodiments, the output 218 can also include control signals configured for communication with an automated drug-delivery device to facilitate continuous, closed-loop insulin management. By encompassing the results of the refined mathematical representation and associated recommendations, the output 218 allows the system 200 to support ongoing, data-driven management of insulin dynamics in real-time.

In one or more embodiments, the output 218 may be further configured to generate one or more clinical recommendations, one or more lifestyle recommendations, and one or more monitoring plans using one or more of an insulin-demand prediction, an insulin-supply value, and an insulin-efficiency optimization produced at least during an instance wherein one or more predictive outputs are generated. The output 218 may combine these predictive components individually or in any suitable combination to present actionable therapeutic guidance, behavioral suggestions, or longitudinal monitoring strategies that reflect the refined mathematical representation of the patient state.

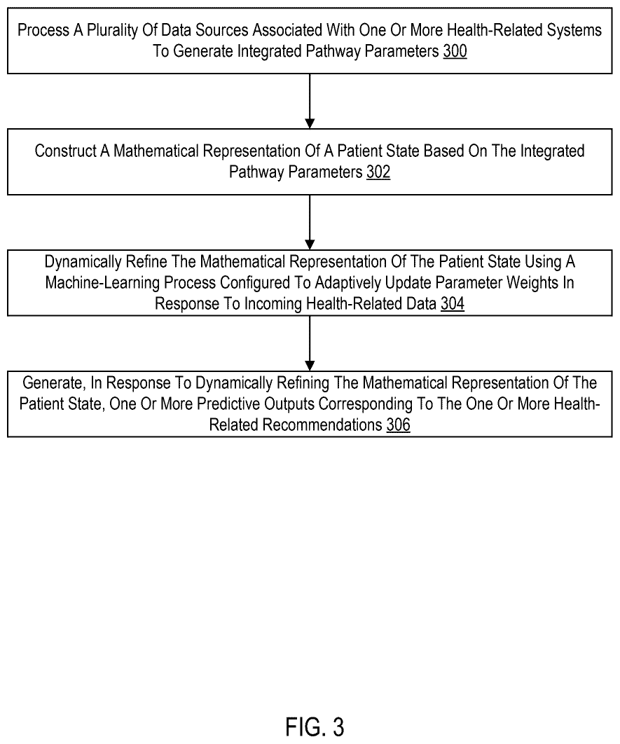

FIG. 2 therefore illustrates an example system architecture configured to perform the processes described in the subsequent figures. The data acquisition component 206, the data discovery component 210, the machine learning layer 212, the mathematical modeling component 214, and the optimization engine 216 cooperate to acquire and process multi-source patient data, identify biologically relevant relationships, construct and refine mathematical representations of patient states, and include predictive outputs corresponding to health-related recommendations. The layered configuration of the system 200 supports continuous adaptation to new data, enabling the system to update and refine model parameters in real-time as additional information becomes available. In one or more embodiments, the system 200 can be implemented within a distributed computing or cloud-based environment that facilitates scalability, secure data exchange, and interoperability with clinical or therapeutic systems. FIG. 3 sets forth a flowchart illustrating an example method executed by the system 200, or the computing system 100, for managing insulin dynamics, including data acquisition, model construction, adaptive refinement, and the inclusion of health-related recommendations in accordance with embodiments of the present disclosure.

For further explanation, FIG. 3 sets forth a flowchart illustrating an example method of managing insulin dynamics and generating one or more health-related recommendations in accordance with embodiments of the present disclosure. The example method of FIG. 3 can be carried out in a system similar to that of FIG. 2. The method of FIG. 3 can be performed by the system 200 (which, in some embodiments, can be representative of the computing system 100) or by one or more components thereof, including a data acquisition component (e.g., the data acquisition component 206), a data discovery component (e.g., the data discovery component 210), a machine learning layer (e.g., the machine learning layer 212), a mathematical modeling component (e.g., the mathematical modeling component 214), and an optimization engine (e.g., the optimization engine 216).

The method of FIG. 3 includes processing 300 a plurality of data sources associated with one or more health-related systems to generate integrated pathway parameters. The processing 300 step may be carried out by obtaining multi-omics profiles, clinical records, and real-time streaming health data from a digital twin database (e.g., the digital twin database 202), as well as biological pathway information from a knowledge graph database (e.g., the knowledge graph database 208), and aggregating the obtained information using the data acquisition component 206. In one or more examples, the processing 300 step may also be carried out by obtaining lifestyle or environmental information such as dietary intake, activity patterns, sleep behavior, stress indicators, or ambient condition measurements. Such information may be acquired through user-entered data, connected applications, or third-party data services in addition to sensor-derived inputs. The processing 300 step may further include normalizing, aligning, and transforming the aggregated data to ensure consistency across heterogeneous sources, followed by analyzing the preprocessed data using the data discovery component 210 to identify relevant biomarkers, pathway interactions, and physiological variables that influence insulin regulation. The results of the processing 300 step may include integrated pathway parameters that capture relationships among molecular, clinical, and temporal factors within a biological system of a patient, forming the basis for downstream modeling and adaptive optimization.

The method of FIG. 3 also includes constructing 302 a mathematical representation of a patient state based on the integrated pathway parameters. The constructing 302 step may be carried out by the mathematical modeling component 214. During the constructing 302 step, the mathematical modeling component 214 can receive the integrated pathway parameters generated from the processing 300 step and translate the integrated pathway parameters into a quantitative model that represents the relationships among insulin demand, supply, and efficiency over time. The constructing 302 step may include formulating a QUBO model or another computational framework configured to encode the interdependencies among biological pathways, metabolic variables, and clinical indicators associated with insulin regulation. The mathematical representation of the patient state produced during the constructing 302 step can serve as a computational model capable of simulating insulin dynamics of the patient under varying physiological and behavioral conditions.

The method of FIG. 3 also includes refining 304 the mathematical representation of the patient state using adaptive computation. The refining 304 step may be carried out by the optimization engine 216. During the refining 304 step, the optimization engine 216 can receive the mathematical representation generated during the constructing 302 step and perform iterative computations to adjust model parameters, validate dependencies, and enhance predictive accuracy. The refining 304 step may include applying constraint-based and machine-learning-assisted optimization techniques that ensure the mathematical representation conforms to physiological, safety, and efficacy parameters. In one or more embodiments, the refining 304 step can involve evaluating the mathematical representation against real-time patient data received through the data acquisition component 206, performing solution validation and constraint checking, as well as executing performance tracking routines to maintain model stability. The refined mathematical representation generated during the refining 304 step can serve as an updated computational model for continuous monitoring and adaptive management of insulin dynamics.

The method of FIG. 3 also includes generating 306 one or more predictive outputs corresponding to health-related recommendations based on the refined mathematical representation of the patient state. The generating 306 may be carried out by the optimization engine 216 in coordination with the machine learning layer 212. During the generating 306 step, the refined mathematical representation produced during the refining 304 step can be evaluated to produce predictive insights, including insulin-demand and supply forecasts, efficiency metrics, and patient-specific recommendations for clinical or lifestyle interventions. The generating 306 step may include quantifying uncertainty through confidence intervals, applying safety bounds to ensure physiologically valid outputs, and identifying atypical data patterns that warrant additional analysis or intervention. In one or more embodiments, the generating 306 step can further include transmitting predictive outputs for presentation through a user interface or for communication to an automated drug-delivery device. The results of the generating 306 step can support continuous, data-driven management of insulin dynamics tailored to individual patient needs.

The method steps of FIG. 3 collectively describe a continuous process for managing insulin dynamics through data integration, mathematical modeling, adaptive refinement, and generation of predictive outputs. The processing 300 step, the constructing 302 step, the refining 304 step, and the generating 306 step together enable the system 200 to acquire and analyze multi-source patient data, model complex biological relationships, and produce actionable, patient-specific recommendations in real-time. In one or more embodiments, the method of FIG. 3 can be executed continuously or periodically to update the mathematical representation of the patient state as new clinical or physiological data becomes available. By coordinating these operations across the data acquisition component 206, the machine learning layer 212, the mathematical modeling component 214, and the optimization engine 216, the method of FIG. 3 facilitates adaptive and reliable management of insulin dynamics in accordance with embodiments of the present disclosure.

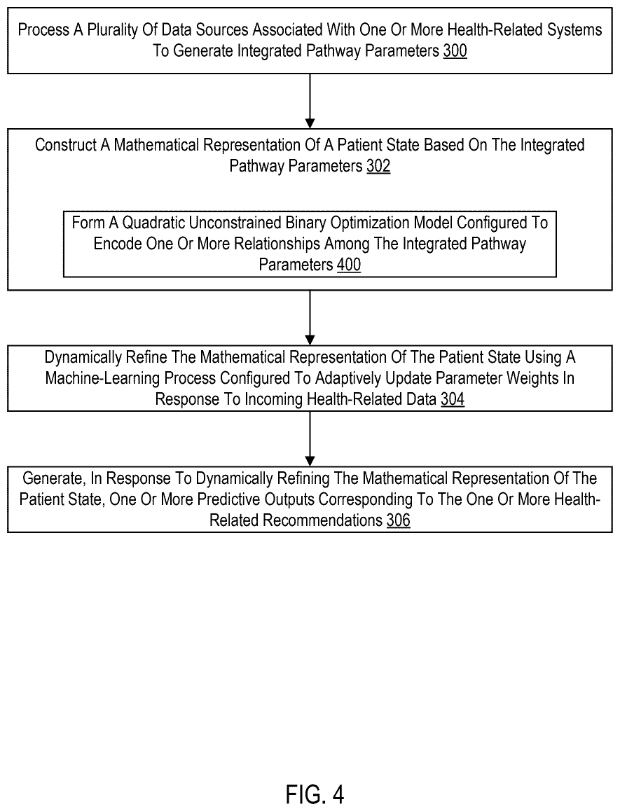

FIG. 3 therefore illustrates an example method that defines the operational flow executed by the system 200 for managing insulin dynamics. The sequence of steps shown in FIG. 3 demonstrates how the system 200 continuously acquires, models, refines, and evaluates patient-specific biological information to generate predictive outputs corresponding to health-related recommendations. The flowchart of FIG. 3 provides a foundation for the subsequent figures, which further expand upon the operations shown. FIG. 4, in particular, sets forth a flowchart illustrating an example method that expands upon the operations described with reference to FIG. 3 by detailing the manner in which the system 200 constructs the mathematical representation of the patient state and performs iterative refinement of the model using adaptive computation in accordance with embodiments of the present disclosure.

For further explanation, FIG. 4 sets forth a flowchart illustrating an example method of constructing and refining a mathematical representation of a patient state in accordance with embodiments of the present disclosure. The example method of FIG. 4 can be carried out in a system similar to that of FIG. 2. The method of FIG. 4 can be performed by the system 200 (which, in some embodiments, can be representative of the computing system 100) or by one or more components thereof, including a machine learning layer (e.g., the machine learning layer 212), a mathematical modeling component (e.g., the mathematical modeling component 214), and an optimization engine (e.g., the optimization engine 216).

The method of FIG. 4 includes forming 400 a QUBO model based on the integrated pathway parameters as part of the constructing 302 step described with reference to FIG. 3. The processing 300 step may be performed as described with reference to FIG. 3 to generate the integrated pathway parameters used during the forming 400 step. The forming 400 step may be carried out by the mathematical modeling component 214. During the forming 400 step, the mathematical modeling component 214 defines binary decision variables corresponding to pathway states or feature selections, specifies an objective function that encodes alignment with patient-specific targets, and assigns linear and quadratic coefficients that quantify associations among biomarkers, clinical indicators, and temporal factors. The forming 400 step may further include mapping physiological or safety requirements into penalty terms, assembling an interaction matrix that captures pairwise dependencies, and calibrating coefficient magnitudes using statistics derived from the integrated pathway parameters. The QUBO model produced during the forming 400 step provides a machine-solvable model that precedes the refining 304 step, in which the optimization engine 216 evaluates and improves candidate solutions. The QUBO model produced during the forming 400 step also precedes the generating 306 step, in which the optimization engine 216 and the machine learning layer 212 produce one or more predictive outputs corresponding to health-related recommendations based on the refined mathematical representation of the patient state.

The method steps of FIG. 4 collectively describe a process performed by the system 200 for constructing 302 a mathematical representation of a patient state and preparing the model for adaptive refinement and output generation. The processing 300 step may be performed as described with reference to FIG. 3 to produce the integrated pathway parameters that serve as the foundation for the forming 400 step, during which the mathematical modeling component 214 encodes the integrated pathway parameters into a QUBO model representative of the biological, clinical, and temporal relationships associated with insulin regulation. The QUBO model formed during the forming 400 step enables the system 200 to transition into the refining 304 step, where the optimization engine 216 iteratively improves candidate solutions, and the generating 306 step, where the optimization engine 216 and the machine learning layer 212 produce predictive outputs corresponding to health-related recommendations.

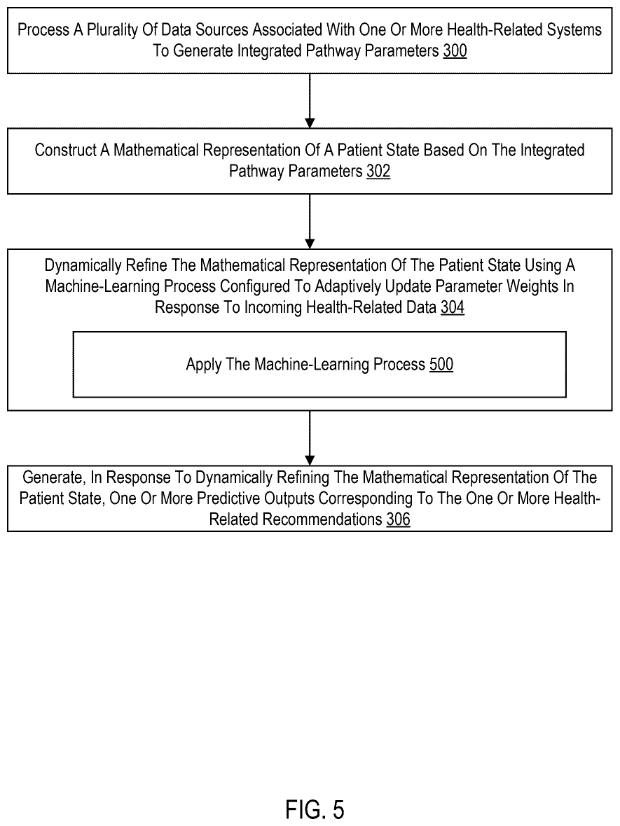

FIG. 4 therefore illustrates an example method that defines the computational framework executed by the system 200 for constructing 302 and preparing to refine 304 a mathematical representation of a patient state. The sequence of operations shown in FIG. 4 demonstrates how the system 200 converts integrated pathway parameters into a machine-solvable model that serves as the foundation for continuous optimization and prediction. FIG. 5 sets forth a flowchart illustrating an example method that expands upon the operations described with reference to FIG. 4 by detailing the manner in which the system 200 performs the refining 304 step to dynamically optimize, update, and validate the mathematical representation of the patient state in accordance with embodiments of the present disclosure.

For further explanation, FIG. 5 sets forth a flowchart illustrating an example method of refining a mathematical representation of a patient state in accordance with embodiments of the present disclosure. The example method of FIG. 5 can be carried out in a system similar to that of FIG. 2. The method of FIG. 5 can be performed by the system 200 (which, in some embodiments, can be representative of the computing system 100) or by one or more components thereof, including a machine learning layer (e.g., the machine learning layer 212), a mathematical modeling component (e.g., the mathematical modeling component 214), and an optimization engine (e.g., the optimization engine 216).

The method of FIG. 5 includes applying 500 a machine-learning process to refine the mathematical representation of the patient state as part of the refining 304 step described with reference to FIG. 3. The processing 300 step and the constructing 302 step may each be performed as is also described with reference to FIG. 3 to provide the data, model structure, and predictive outputs associated with the applying 500 step. The applying 500 step may be carried out by the machine learning layer 212 in coordination with the optimization engine 216. During the applying 500 step, the machine learning layer 212 can implement adaptive learning algorithms, such as graph neural networks, reinforcement learning models, neural-network prediction models, or genetic-algorithm optimization routines, to continuously update model parameters based on newly acquired data. The applying 500 step may include training and adjusting the QUBO model using real-time inputs received from the data acquisition component 206, re-weighting relationships among the integrated pathway parameters, and modifying the coefficients of the QUBO model to improve alignment with observed physiological responses. In some embodiments, the applying 500 step can further include incorporating feedback from previous optimization cycles, validating model predictions against known clinical outcomes, and performing iterative learning to enhance predictive accuracy. In one or more embodiments, the refined mathematical representation produced during the applying 500 step can serve as the input for the optimization procedures executed during the refining 304 step and for generating one or more predictive outputs corresponding to health-related recommendations during the generating 306 step as is further described with reference to FIG. 3.

The method steps of FIG. 5 collectively describe operations performed by the system 200 for refining 304 a mathematical representation of a patient state using adaptive machine-learning processes. The processing 300 step, the constructing 302 step, and the generating 306 step may each be performed as described with reference to FIG. 3, while the applying 500 step represents the portion of the refining 304 step in which the machine learning layer 212 and the optimization engine 216 implement adaptive learning algorithms to continuously update model parameters, variable weightings, and interaction coefficients based on real-time and historical patient data. The operations of FIG. 5 enable the system 200 to improve the precision and responsiveness of the mathematical representation, allowing the model to evolve dynamically with each iteration of learning and optimization.

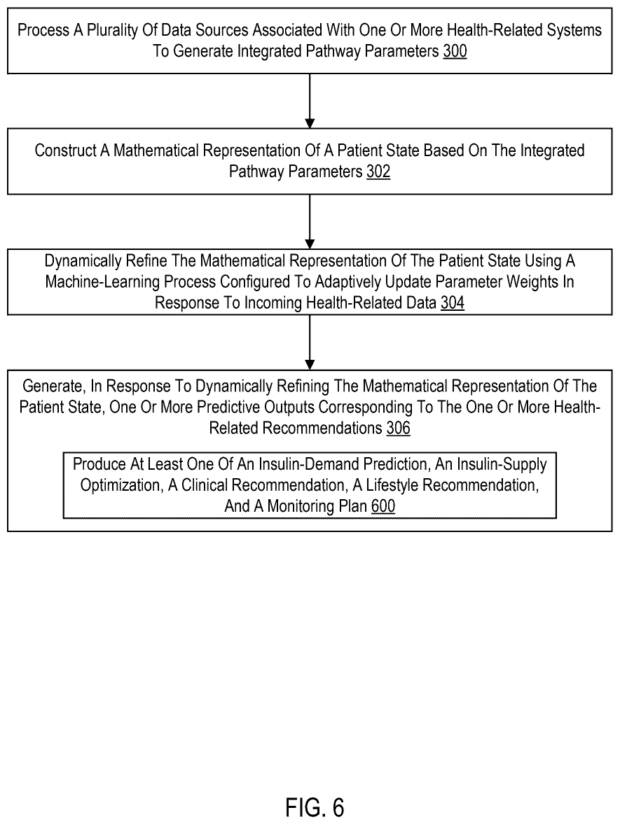

FIG. 5 therefore illustrates an example method executed by the system 200 for refining 304 a mathematical representation of a patient state through the application of a machine-learning process. The sequence of operations shown in FIG. 5 demonstrates how the system 200 transitions from the formulation of a static model to an adaptive computational framework capable of real-time optimization and continuous learning. FIG. 6 sets forth a flowchart illustrating an example method that expands upon the operations described with reference to FIG. 5 by detailing how the system 200 performs the generating 306 step to produce predictive outputs corresponding to health-related recommendations in accordance with embodiments of the present disclosure.

For further explanation, FIG. 6 sets forth a flowchart illustrating an example method of generating predictive outputs corresponding to health-related recommendations in accordance with embodiments of the present disclosure. The example method of FIG. 6 can be carried out in a system similar to that of FIG. 2. The method of FIG. 6 can be performed by the system 200 (which, in some embodiments, can be representative of the computing system 100) or by one or more components thereof, including a machine learning layer (e.g., the machine learning layer 212) and an optimization engine (e.g., the optimization engine 216).

The method of FIG. 6 includes producing 600 one or more predictive outputs corresponding to health-related recommendations as part of the generating 306 step described with reference to FIG. 3. The processing 300 step, the constructing 302 step, and the refining 304 step may each be performed as is also described with reference to FIG. 3 to provide the integrated data, mathematical representation, and refined model utilized during the producing 600 step. The producing 600 step may be carried out by the optimization engine 216 in coordination with the machine learning layer 212. During the producing 600 step, the optimization engine 216 can evaluate the refined mathematical representation of the patient state generated during the refining 304 step to determine predictive insights related to insulin dynamics. The producing 600 step may include generating insulin-demand and supply forecasts, identifying pathway efficiency metrics, and developing patient-specific clinical or lifestyle recommendations that account for real-time and historical physiological data. In one or more embodiments, the producing 600 step can further include applying confidence thresholds, physiological safety bounds, and comparative validations to ensure that the generated outputs are reliable and clinically relevant. The results of the producing 600 step can be communicated for presentation through a clinical user interface or transmitted to an automated therapeutic control system to enable continuous, adaptive management of insulin dynamics.

The method steps of FIG. 6 collectively describe operations performed by the system 200 for generating 306 one or more predictive outputs corresponding to health-related recommendations. The processing 300 step, the constructing 302 step, and the refining 304 step may each be performed as described with reference to FIG. 3, while the producing 600 step represents the stage at which the optimization engine 216 and the machine learning layer 212 translate the refined mathematical representation of the patient state into actionable, patient-specific outputs. The operations of FIG. 6 enable the system 200 to convert optimized computational results into clinically interpretable recommendations, facilitating real-time decision support and automated therapeutic control.

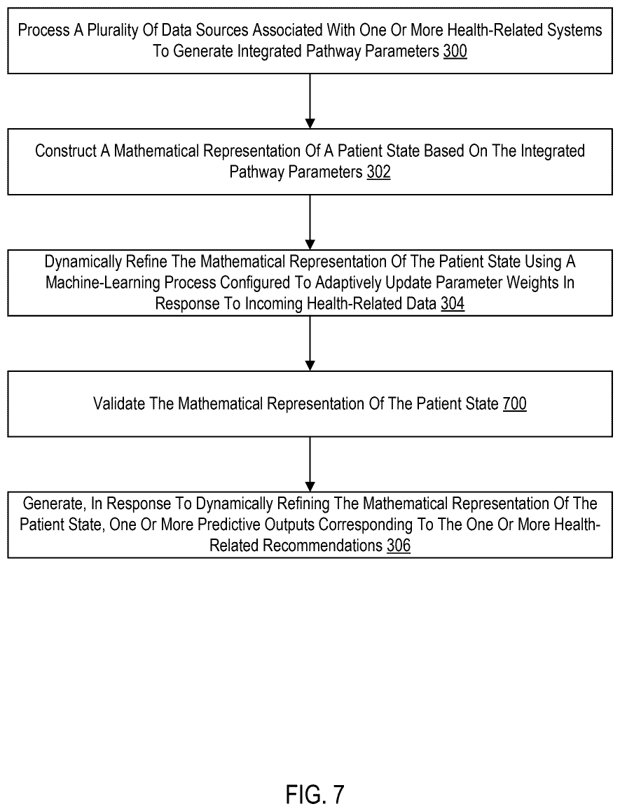

FIG. 6 therefore illustrates an example method executed by the system 200 for generating 306 one or more predictive outputs corresponding to health-related recommendations. The sequence of operations shown in FIG. 6 demonstrates how the system 200 uses the refined mathematical representation of the patient state to produce validated, personalized outputs that inform both clinical and automated responses. FIG. 7 sets forth a flowchart illustrating an example method that expands upon the operations described with reference to FIGS. 3-6 by detailing the manner in which the system 200 performs validation of the mathematical representation of the patient state to ensure consistency with physiological, safety, and efficacy parameters in accordance with embodiments of the present disclosure.

For further explanation, FIG. 7 sets forth a flowchart illustrating an example method of validating a mathematical representation of a patient state in accordance with embodiments of the present disclosure. The example method of FIG. 7 can be carried out in a system similar to that of FIG. 2. The method of FIG. 7 can be performed by the system 200 (which, in some embodiments, can be representative of the computing system 100) or by one or more components thereof, including an optimization engine (e.g., the optimization engine 216) and a machine learning layer (e.g., the machine learning layer 212).

The method of FIG. 7 includes validating 700 the mathematical representation of the patient state as an operation performed after the refining 304 step and before the generating 306 step described with reference to FIG. 3. The processing 300 step, the constructing 302 step, the refining 304 step, and the generating 306 step may each be performed as described with reference to FIG. 3 to provide the integrated data, mathematical representation, and refined outputs utilized during the validating 700 step. The validating 700 step may be carried out by the optimization engine 216 in coordination with the machine learning layer 212. During the validating 700 step, the optimization engine 216 can assess the refined mathematical representation to ensure that the encoded parameters, constraints, and relationships remain consistent with physiological, safety, and efficacy requirements. The validating 700 step may include evaluating variable dependencies, verifying model convergence, and confirming that optimization results align with biological and clinical expectations. In one or more embodiments, the validating 700 step can further include performing constraint checks, sensitivity analyses, and comparative evaluations against real-time or historical patient data to confirm that the mathematical representation remains accurate and stable for ongoing use. The validated mathematical representation produced during the validating 700 step can then serve as a verified computational framework for the generating 306 step, ensuring the reliability of predictive outputs corresponding to health-related recommendations.

The method steps of FIG. 7 collectively describe operations performed by the system 200 for validating 700 the mathematical representation of a patient state to ensure consistency with physiological, safety, and efficacy parameters. The processing 300 step, the constructing 302 step, the refining 304 step, and the generating 306 step may each be performed as described with reference to FIG. 3, while validating 700 represents the stage at which the optimization engine 216 and the machine learning layer 212 evaluate the refined mathematical representation to confirm the accuracy, stability, and biological relevance of the refined mathematical representation. The operations of FIG. 7 enable the system 200 to verify that the optimized model reflects clinically acceptable relationships between insulin demand, supply, and efficiency before generating health-related recommendations.

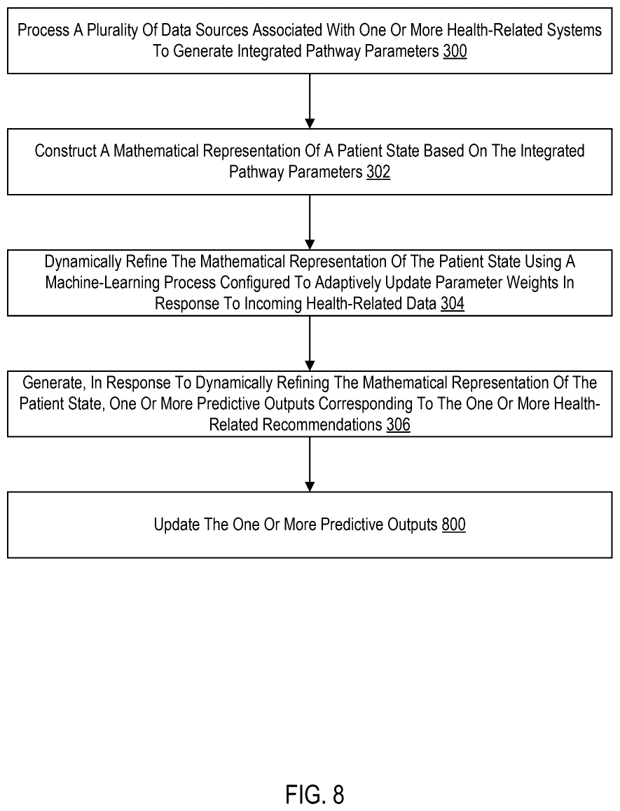

FIG. 7 therefore illustrates an example method executed by the system 200 for validating 700 a mathematical representation of a patient state. The sequence of operations shown in FIG. 7 demonstrates how the system 200 ensures that the refined mathematical representation produced during the refining 304 step satisfies predefined constraints and physiological expectations, thereby supporting reliable and safe operation of subsequent processes. FIG. 8 sets forth a flowchart illustrating an example method that expands upon the operations described with reference to FIGS. 3-7 by detailing the manner in which the system 200 performs iterative optimization and model enhancement procedures to improve predictive performance in accordance with embodiments of the present disclosure.

For further explanation, FIG. 8 sets forth a flowchart illustrating an example method of performing iterative optimization and model enhancement to improve predictive performance in accordance with embodiments of the present disclosure. The example method of FIG. 8 can be carried out in a system similar to that of FIG. 2. The method of FIG. 8 can be performed by the system 200 (which, in some embodiments, can be representative of the computing system 100) or by one or more components thereof, including a machine learning layer (e.g., the machine learning layer 212) and an optimization engine (e.g., the optimization engine 216).

The method of FIG. 8 includes updating 800 one or more predictive outputs based on real-time streaming health data associated with one or more patients after the generating 306 step described with reference to FIG. 3. The processing 300 step, the constructing 302 step, the refining 304 step, and the generating 306 step may each be performed as described with reference to FIG. 3 to provide the integrated data, mathematical representation, and initial predictive outputs utilized during the updating 800 step. The updating 800 step may be carried out by the optimization engine 216 in coordination with the machine learning layer 212. During the updating 800 step, the optimization engine 216 can continuously evaluate and revise predictive outputs using new data received through the data acquisition component 206 from wearable sensors, continuous glucose monitoring devices, or electronic medical record systems. The updating 800 step may include recalculating insulin-demand and supply forecasts, adjusting pathway efficiency metrics, and modifying clinical or lifestyle recommendations to reflect changes in physiological conditions or behavioral patterns. In one or more embodiments, the updating 800 step can further include applying confidence thresholds and safety constraints to ensure the accuracy and reliability of the updated outputs. The predictive outputs produced during the updating 800 step enable the system 200 to provide adaptive, data-driven recommendations that reflect current patient conditions and support continuous, personalized management of insulin dynamics.

The method steps of FIG. 8 collectively describe operations performed by the system 200 for updating 800 one or more predictive outputs corresponding to health-related recommendations based on real-time streaming health data. The processing 300 step, the constructing 302 step, the refining 304 step, and the generating 306 step may each be performed as described with reference to FIG. 3, while updating 800 represents the stage at which the optimization engine 216 and the machine learning layer 212 incorporate new data received from wearable sensors, glucose monitors, or electronic medical records to revise previously generated outputs. The operations of FIG. 8 enable the system 200 to maintain the accuracy and clinical relevance of the health-related recommendations by continuously aligning predictive outputs with the most current patient information and environmental factors.

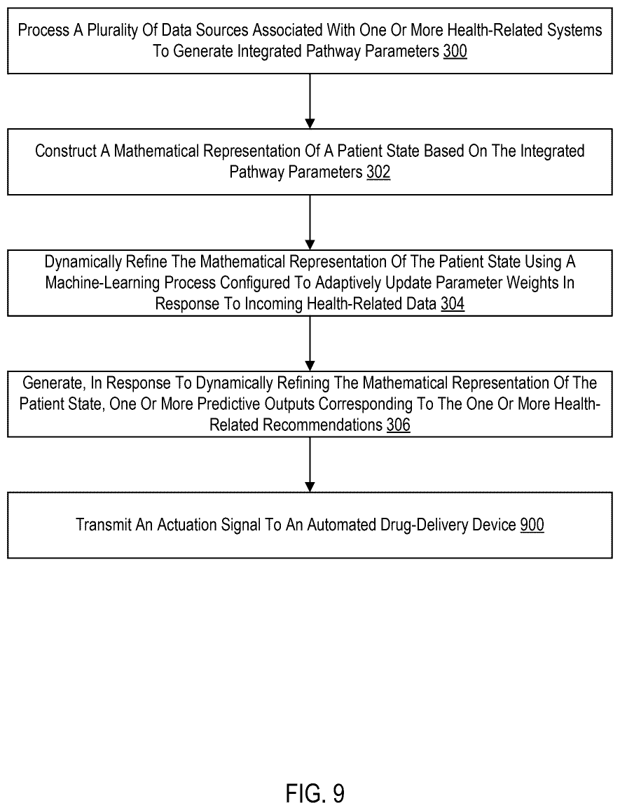

FIG. 8 therefore illustrates an example method executed by the system 200 for updating 800 one or more predictive outputs corresponding to health-related recommendations. The sequence of operations shown in FIG. 8 demonstrates how the system 200 leverages real-time streaming data to dynamically adjust predictive forecasts, clinical insights, and personalized therapeutic guidance, ensuring that each recommendation remains timely, physiologically consistent, and reflective of the current state of the patient. FIG. 9 sets forth a flowchart illustrating an example method that expands upon the operations described with reference to FIGS. 3-8 by detailing the manner in which the system 200 performs automated control actions based on updated predictive outputs to facilitate closed-loop insulin management in accordance with embodiments of the present disclosure.

For further explanation, FIG. 9 sets forth a flowchart illustrating an example method of transmitting an actuation signal to an automated drug-delivery device based on one or more predictive outputs corresponding to health-related recommendations in accordance with embodiments of the present disclosure. The example method of FIG. 9 can be carried out in a system similar to that of FIG. 2. The method of FIG. 9 can be performed by the system 200 (which, in some embodiments, can be representative of the computing system 100) or by one or more components thereof, including an optimization engine (e.g., the optimization engine 216) and a machine learning layer (e.g., the machine learning layer 212).

The method of FIG. 9 includes transmitting 900 an actuation signal to an automated drug-delivery device based on predictive outputs corresponding to health-related recommendations as part of the output 218 described with reference to FIG. 2. The processing 300 step, the constructing 302 step, the refining 304 step, and the generating 306 step may each be performed as described with reference to FIG. 3 to provide the integrated data, refined model, and predictive outputs utilized during the transmitting 900 step. The transmitting 900 step may be carried out by the optimization engine 216 in coordination with the machine learning layer 212. During the transmitting 900 step, the optimization engine 216 can convert the predictive outputs generated during the generating 306 step into an actuation signal for the automated drug-delivery device. The transmitting 900 step may include encoding therapeutic parameters, such as dosage level, administration timing, or delivery duration, into the actuation signal based on patient-specific conditions and safety constraints. In one or more embodiments, the transmitting 900 step can further include verifying that the actuation signal complies with physiological and regulatory thresholds, confirming secure communication with the automated drug-delivery device, and monitoring feedback signals to ensure accurate execution of the therapeutic action. The transmitting 900 step enables the system 200 to implement automated, closed-loop insulin management by transforming predictive outputs into precise therapeutic control instructions.

The method steps of FIG. 9 collectively describe operations performed by the system 200 for transmitting 900 an actuation signal to an automated drug-delivery device based on predictive outputs corresponding to health-related recommendations. The processing 300 step, the constructing 302 step, the refining 304 step, and the generating 306 step may each be performed as described with reference to FIG. 3, while transmitting 900 represents the stage at which the optimization engine 216 and the machine learning layer 212 convert the predictive outputs of the system into executable control instructions. The operations of FIG. 9 enable the system 200 to achieve closed-loop automation by linking computationally derived recommendations directly to therapeutic delivery, ensuring that each action aligns with patient-specific physiological conditions and safety constraints.

FIG. 9 therefore illustrates an example method executed by the system 200 for transmitting 900 an actuation signal to an automated drug-delivery device. The sequence of operations shown in FIG. 9 demonstrates how the system 200 transforms continuously refined predictive outputs into automated therapeutic actions that maintain real-time glycemic control and patient safety. Although FIG. 9 refers to automated drug-delivery devices as an example, the actuation process described herein may also be applied to other automated or assistive systems, including, but not limited to, infusion pumps, neural-stimulation devices, ventilators, cardiac pacing systems, environmental control mechanisms, or user-operated devices such as smartphones, tablets, or wearable systems configured to provide and/or receive notifications, alerts, or control actions based on predictive outputs. FIG. 10 sets forth a block diagram illustrating an example cloud computing environment suitable for implementing one or more embodiments of the present disclosure. The environment of FIG. 10 further depicts how the system 200 and computing system 100 may be deployed within distributed or hybrid infrastructures to support scalable, secure, and continuous management of insulin dynamics.

For further explanation, FIG. 10 sets forth a block diagram of a cloud computing environment suitable for implementing one or more embodiments of the present disclosure. The cloud computing environment of FIG. 10 may be used to deploy and manage the system 200 and the computing system 100 for continuous, adaptive management of insulin dynamics. As shown in FIG. 10, the cloud service provider 1002 can deliver computing, platform, and software resources through a service-based consumption model in which resources are provisioned on demand and accessed as managed services. One or more clients 1032 may access the cloud service provider 1002 through a network 1034, which may include the Internet, a private medical network, or a hybrid infrastructure that supports secure and efficient data exchange between distributed systems, healthcare providers, and end-user devices. The cloud service provider 1002 may operate within a public, private, or hybrid cloud configuration, ensuring reliable communication, scalability, and interoperability across the distributed components of the environment.

FIG. 10 depicts an embodiment in which software 1020 is delivered as a service.

Software-as-a-Service (SaaS) provides access to software applications hosted by the cloud service provider 1002 over the network 1034, eliminating the need for local installation or maintenance within clinical or research environments. As examples of the software 1020 delivered as a service, the illustrated embodiment includes office productivity 1022 software, customer relationship management (CRM) 1024 software, and project management 1026 software. In the context of managing insulin dynamics, additional software services may include clinical data management applications, analytics dashboards for visualizing patient outcomes, and AI-based modeling platforms that interface with the system 200 and the computing system 100. The software 1020 resources may allow healthcare providers, researchers, and connected medical devices to securely interact with the cloud-hosted systems to generate, review, and apply real-time health-related recommendations.

FIG. 10 also depicts an embodiment in which platform 1012 resources are delivered as a service. Platform-as-a-Service (PaaS) provides managed environments that allow developers, healthcare organizations, and research teams to build, deploy, and scale data-driven applications for managing insulin dynamics without maintaining the underlying infrastructure. As examples of the platform 1012 resources, the illustrated embodiment includes database 1014 services, development tools 1016 services, and execution runtime 1018 services. The database 1014 services may provide scalable, secure data storage used to maintain multi-omics datasets, clinical records, and real-time sensor data associated with the digital twin database 202. The development tools 1016 services may include integrated environments for building and testing applications that utilize the system 200 and the computing system 100 for generating predictive outputs and health-related recommendations. The execution runtime 1018 services may provide managed computing resources capable of executing optimization and machine learning workloads, including the adaptive modeling and QUBO processing operations performed by the mathematical modeling component 214 and the optimization engine 216.

FIG. 10 further depicts an embodiment in which infrastructure 1004 resources are delivered as a service. Infrastructure-as-a-Service (IaaS) provides virtualized computing hardware resources that include compute 1006, storage 1008, and networking 1010 capabilities. The compute 1006 resources may include virtual machines, containers, or specialized accelerators such as graphics processing units (GPUs) or tensor processing units (TPUs) configured to perform optimization, training, and inference operations associated with the system 200 and the computing system 100. The storage 1008 resources may include scalable block or object storage configured to securely maintain datasets, digital twin records, and knowledge graph information used by the data acquisition component 206 and the data discovery component 210. The networking 1010 resources may include virtual networks, secure gateways, or virtual private clouds (VPCs) that provide reliable, encrypted communication channels between distributed system components, external clinical systems, and connected medical devices. Together, the infrastructure 1004 resources enable the deployment of scalable, high-performance computing environments that support continuous, data-driven management of insulin dynamics.

FIG. 10 further depicts an embodiment in which the cloud service provider 1002 delivers security 1028 and management 1030 resources as part of the overall service architecture. The security 1028 resources may include encryption, authentication, identity management, and monitoring services that safeguard patient data, model parameters, and communications across the distributed components of the system 200 and the computing system 100. The management 1030 resources may include administrative consoles, orchestration frameworks, and automated scaling policies that enable dynamic provisioning and performance optimization of the cloud-based environment. In one or more embodiments, the management 1030 resources can also coordinate the deployment of machine learning workloads, synchronization of the digital twin database 202 and the knowledge graph database 208, as well as real-time adjustment of data acquisition, modeling, and optimization processes to maintain continuous, adaptive operation. These resources collectively ensure secure, compliant, and efficient execution of the systems and methods for managing insulin dynamics in accordance with embodiments of the present disclosure.

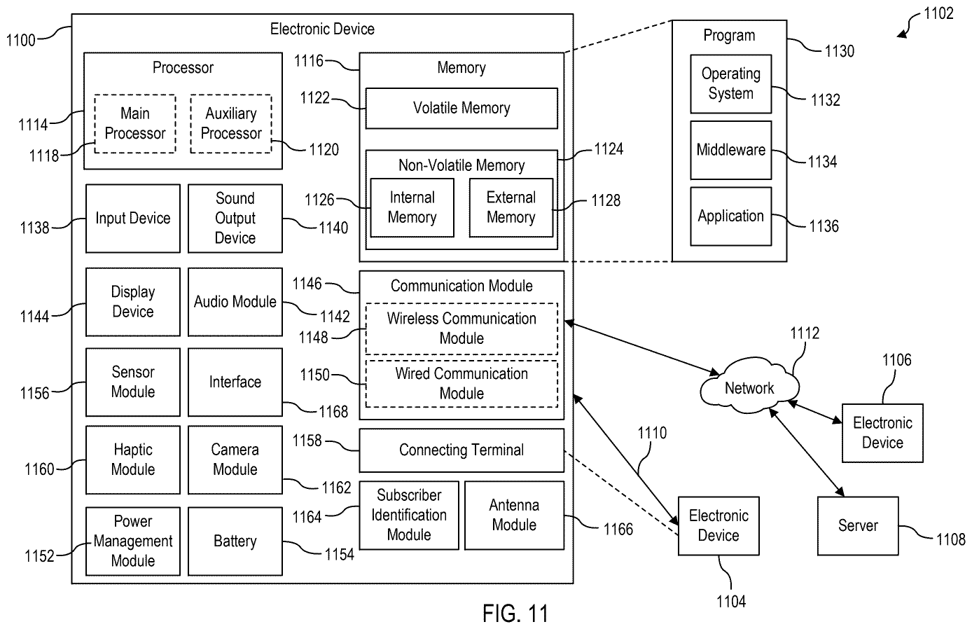

FIG. 10 therefore illustrates a cloud-based computing environment configured to support deployment, operation, and scaling of the system 200 and the computing system 100 for managing insulin dynamics. The arrangement shown in FIG. 10 demonstrates how the cloud service provider 1002 delivers a unified framework in which the infrastructure 1004 resources, the platform 1012 resources, and the software 1020 resources cooperate to provide continuous data processing, adaptive modeling, and secure delivery of predictive outputs. By leveraging the cloud service provider 1002, healthcare organizations and connected devices can perform distributed machine learning, optimization, and control tasks without the limitations of on-premises hardware and in real-time. FIG. 11 sets forth a block diagram illustrating an example electronic device suitable for implementing one or more components of the system 200 or the computing system 100, providing the processing, storage, and communication resources required to execute the operations described throughout the present disclosure.

FIG. 11 is a block diagram of an electronic device 1100 in a network environment 1102 in accordance with embodiments of the present disclosure. The electronic device 1100 may operate independently or in conjunction with other electronic devices 1104 and 1106, or a server 1108, through a first network 1110 (e.g., a short-range communication network) or a second network 1112 (e.g., a long-range communication network). The electronic device 1100 may correspond to, or include, the functional components of the computing system 100 or the system 200 described with reference to FIGS. 1 and 2. For example, the electronic device 1100 may execute one or more functionalities associated with the data acquisition component 206, the data discovery component 210, the machine learning layer 212, the mathematical modeling component 214, and the optimization engine 216 to perform one or more operations associated with the management of insulin dynamics, including data processing, model construction, adaptive refinement, validation, predictive output generation, and automated control.

Referring to FIG. 11, the components of the electronic device 1100 illustrated therein will now be described in additional detail. These components may collectively enable the electronic device 1100 to execute the systems and methods described throughout this disclosure, including at least the processing 300 step, the constructing 302 step, the refining 304 step, the validating 700 step, the generating 306 step, the updating 800 step, and the transmitting 900 step associated with the continuous management of insulin dynamics. While particular components are shown in FIG. 11, additional or alternative components may be included in other embodiments, and the described components may be implemented as discrete hardware modules, integrated circuits, or combinations thereof.

A processor 1114 may control overall operation of the electronic device 1100 and execute instructions stored in a memory 1116 to perform insulin management operations. The processor 1114 may include a main processor 1118 and an auxiliary processor 1120 that operate independently or cooperatively to manage computational and communication tasks. The main processor 1118 may execute high-level operations of the system 200, including data integration, adaptive modeling, and optimization routines associated with insulin regulation. The auxiliary processor 1120 may perform specialized functions, such as real-time monitoring, communication management, or data synchronization with external devices or cloud services. In some embodiments, the auxiliary processor 1120 may continue to operate while the main processor 1118 is in a low-power state, maintaining network connectivity, processing incoming health data, or performing background learning updates to ensure uninterrupted management of insulin dynamics.

The memory 1116 may include both volatile memory 1122 and non-volatile memory 1124 configured to store data and instructions used by the processor 1114 during operation of the electronic device 1100. The non-volatile memory 1124 may include internal memory 1126 and external memory 1128 that store persistent datasets, software modules, and configuration parameters used for executing insulin management functions. The memory 1116 may also store a program 1130 that can include an operating system 1132, middleware 1134, and one or more applications 1136 executed by the processor 1114 to perform the operations associated with the management of insulin dynamics. The program 1130 may include instructions for executing one or more functionalities associated with the data acquisition component 206, the machine learning layer 212, the mathematical modeling component 214, and the optimization engine 216. In some embodiments, the memory 1116 may also cache real-time data streams received from external devices, such as continuous glucose monitors or wearable sensors, enabling efficient retrieval and immediate use during modeling and optimization processes.

An input device 1138 may receive user input, control commands, or external data during operation of the electronic device 1100. The input device 1138 may include a touchscreen, keyboard, mouse, microphone, or other input mechanisms that enable a user or clinician to enter patient data, modify model parameters, or adjust system settings related to insulin management.

In one or more embodiments, the input device 1138 may also include specialized medical interfaces for receiving data directly from external monitoring devices, such as continuous glucose monitors, insulin pumps, or wearable activity sensors. The input device 1138 may further support voice commands or gesture-based inputs to facilitate hands-free operation in clinical or personal health environments.

A sound output device 1140 may output audio signals generated by the electronic device 1100. The sound output device 1140 may include one or more speakers, receivers, or other audio transducers configured to provide notifications, alerts, or audio feedback to the user during insulin management operations. In some embodiments, the sound output device 1140 may issue alerts when glucose levels exceed defined thresholds, when a data synchronization or optimization cycle is complete, or when the automated drug-delivery device requires attention. The sound output device 1140 may operate in conjunction with an audio module 1142 to enable voice communication, audio playback, or audible prompts that assist users in monitoring real-time insulin recommendations or system performance within the network environment 1102.

A display device 1144 may visually present information generated by the processor 1114 to a user of the electronic device 1100. The display device 1144 may include a flat-panel display, touchscreen display, or projection-based display configured to render graphical interfaces and data visualizations related to insulin dynamics management. In one or more embodiments, the display device 1144 may provide a GUI that allows clinicians or patients to view insulin-demand and supply forecasts, pathway efficiency metrics, or patient-state models generated by the machine learning layer 212 and the optimization engine 216. The display device 1144 may also present alerts, trend charts, or recommendations derived from the predictive outputs of the generating 306 step and the updating 800 step. In some embodiments, the display device 1144 may enable interactive navigation of model parameters or treatment scenarios, allowing a user to simulate how behavioral or dietary changes could affect insulin regulation over time.

A communication module 1146 may enable the electronic device 1100 to transmit and receive data through the first network 1110 or the second network 1112. The communication module 1146 may include a wireless communication module 1148 and a wired communication module 1150, which may operate independently or cooperatively to support various communication interfaces. The wireless communication module 1148 may support technologies such as Wi-Fi, Bluetooth, near-field communication (NFC), or cellular connectivity to facilitate real-time data transmission between the electronic device 1100, wearable sensors, cloud-based services, and remote clinical systems. The wired communication module 1150 may provide secure, high-speed data transfer through physical connections such as USB or Ethernet, enabling synchronization with local databases or integration with hospital information systems. In one or more embodiments, the communication module 1146 may also coordinate the secure exchange of data between the digital twin database 202, the knowledge graph database 208, and other external systems to ensure continuous model updates, validation, and real-time alignment of patient-state information.

A power management module 1152 may regulate power distribution and consumption among the components of the electronic device 1100. The power management module 1152 may monitor voltage and current levels supplied to the processor 1114, the memory 1116, the communication module 1146, and other subsystems to maintain stable and efficient operation during continuous data processing and wireless communication. The power management module 1152 may operate in conjunction with a battery 1154, which may supply power to the electronic device 1100 through a rechargeable or replaceable power source. In some embodiments, the power management module 1152 may dynamically adjust power allocation based on computational workload, data transmission frequency, or battery capacity to extend operational life. The power management module 1152 may also implement safety protocols to ensure uninterrupted insulin management functions, such as maintaining critical data storage and communication during transitions between power states or while recharging the battery 1154.

The battery 1154 may provide electrical power to one or more components of the electronic device 1100 under the control of the power management module 1152. The battery 1154 may be implemented as a rechargeable secondary cell, such as a lithium-ion or lithium-polymer battery, or as a replaceable primary cell. The battery 1154 may supply the necessary voltage and current required for continuous execution of insulin management operations, including the processing of health-related data, real-time communication with cloud-based systems, and operation of wearable or connected therapeutic devices. In one or more embodiments, the battery 1154 may include a backup or reserve mode that preserves stored data and operational states within the memory 1116 in the event of power interruption, ensuring consistent management of insulin dynamics and uninterrupted data integrity during system operation.

A sensor module 1156 may detect physiological, environmental, or operational conditions and generate corresponding signals for processing by the processor 1114. The sensor module 1156 may include biosensors, accelerometers, temperature sensors, or pressure sensors that monitor patient activity, ambient conditions, or device performance. In one or more embodiments, the sensor module 1156 may collect physiological data such as glucose levels, heart rate, or skin temperature from the patient and transmit that information to the data acquisition component 206 for integration with other clinical or lifestyle data. The sensor module 1156 may also monitor environmental factors, such as temperature or humidity, to ensure that the electronic device 1100 and connected medical devices operate within safe parameters. In some embodiments, the sensor module 1156 may interface directly with wearable sensors or external monitoring systems through the communication module 1146 to provide continuous data acquisition and adaptive insulin regulation.

A connecting terminal 1158 may include one or more physical connectors that enable the electronic device 1100 to interface with external equipment or peripheral devices. The connecting terminal 1158 may support wired communication standards such as USB, HDMI, or proprietary medical connectors to facilitate data transfer, diagnostic access, or device configuration. In one or more embodiments, the connecting terminal 1158 may provide a secure interface for connecting the electronic device 1100 to external glucose monitoring systems, insulin pumps, or docking stations for charging and data synchronization. The connecting terminal 1158 may also enable communication with hospital infrastructure or laboratory systems, allowing healthcare providers to upload patient data, download updated treatment protocols, or perform remote calibration of insulin management algorithms executed by the system 200.

A haptic module 1160 may provide tactile feedback to a user of the electronic device 1100 during operation. The haptic module 1160 may include one or more actuators, vibration motors, or pressure-responsive components configured to generate physical sensations that correspond to alerts, notifications, or user interactions. In one or more embodiments, the haptic module 1160 may provide vibration alerts to signal abnormal glucose levels, confirm successful data transmission, or indicate completion of an optimization or validation cycle. The haptic module 1160 may also operate in conjunction with the display device 1144 and the sound output device 1140 to deliver multimodal feedback, enhancing user awareness and interaction with the insulin management system. In certain embodiments, the haptic module 1160 may be used to provide discrete, non-auditory alerts for users in clinical or public environments where silent operation is preferred.

A camera module 1162 may capture still images or video data that support the operation and monitoring of the electronic device 1100. The camera module 1162 may include one or more image sensors, lenses, and optical processors configured to record visual information related to the operating environment of the device or the physiological state of the user. In one or more embodiments, the camera module 1162 may facilitate telemedicine functionality by transmitting live video or images to clinicians for remote consultation or assessment of patient compliance. The camera module 1162 may also capture reference images for calibration of connected sensors or documentation of device maintenance events. In some embodiments, the camera module 1162 may operate in coordination with the processor 1114 and the communication module 1146 to perform facial recognition for secure authentication or to monitor user engagement during interactive insulin management sessions.

A subscriber identification module 1164 may store authentication credentials, user-specific information, or subscription data utilized by the communication module 1146 to identify and authorize the electronic device 1100 within the first network 1110 or the second network 1112. The subscriber identification module 1164 may include a secure element, such as a SIM card, eSIM, or embedded cryptographic processor, configured to manage encryption keys and access tokens for secure communication with cloud services, medical networks, or connected healthcare devices. In one or more embodiments, the subscriber identification module 1164 may enable multi-user authentication, ensuring that access to insulin management data, predictive outputs, and control functions is restricted to verified patients, clinicians, or authorized system administrators. The subscriber identification module 1164 may also support remote provisioning or over-the-air updates to maintain compliance with evolving security and healthcare data protection standards.

An antenna module 1166 may enable wireless transmission and reception of signals between the electronic device 1100 and external systems through the communication module 1146. The antenna module 1166 may include one or more antennas configured to support multiple communication protocols, such as Wi-Fi, Bluetooth, cellular, or satellite networks. In one or more embodiments, the antenna module 1166 may facilitate real-time communication with wearable sensors, automated drug-delivery devices, or cloud-based computing systems hosting the digital twin database 202 and the knowledge graph database 208. The antenna module 1166 may be optimized for low-latency, high-bandwidth communication, ensuring reliable transfer of health data and predictive outputs between the electronic device 1100 and remote clinical environments. In some embodiments, the antenna module 1166 may include diversity or beamforming antennas designed to maintain stable connectivity and minimize signal interference during continuous insulin management operations.

An interface 1168 may support communication and data exchange between the electronic device 1100 and external peripherals, systems, or networks. The interface 1168 may include hardware and software components that facilitate input and output operations through wired or wireless communication protocols. In one or more embodiments, the interface 1168 may provide integration with hospital information systems, laboratory databases, or third-party analytics platforms to enable synchronized access to patient records, clinical metrics, and treatment outcomes. The interface 1168 may also allow the electronic device 1100 to connect with wearable sensors, external storage devices, or diagnostic tools for data import and export. In certain embodiments, the interface 1168 may include an API layer that enables interoperability between the system 200, the computing system 100, and external medical software, ensuring seamless data flow, regulatory compliance, and coordinated operation within the broader healthcare network.

FIG. 11 therefore illustrates an example electronic device 1100 and network environment 1102 configured to execute the systems and methods for managing insulin dynamics described herein. The arrangement of components shown in FIG. 11 demonstrates how the system 200 and the computing system 100 may be implemented across mobile, clinical, or distributed computing environments to enable continuous monitoring, adaptive modeling, and automated therapeutic control. The described configuration provides the hardware foundation for performing the operations associated with at least the processing 300 step, the constructing 302 step, the refining 304 step, the validating 700 step, the generating 306 step, the updating 800 step, and the transmitting 900 step described throughout this disclosure. Collectively, FIGS. 1-11 illustrate a comprehensive computing and communication framework that supports secure, scalable, and intelligent management of insulin dynamics in accordance with embodiments of the present disclosure.

In view of the explanations set forth above, at least one skilled in the art will recognize that the benefits of managing insulin dynamics according to embodiments of the present disclosure include:
Improving the operation of the computing system by enabling real-time integration of multi-omics, clinical, and streaming physiological data without requiring manual synchronization, thereby reducing latency and improving system efficiency.
Improving the operation of the computing system by dynamically constructing and refining a mathematical representation of a patient state, enabling adaptive modeling that continuously updates in response to new biological and environmental inputs.
Improving the operation of the computing system by applying machine-learning techniques, such as graph neural networks and reinforcement learning models, to identify correlations and pathway dependencies that enhance predictive accuracy and clinical interpretability.
Improving the operation of the computing system by utilizing a QUBO model to efficiently encode and solve complex relationships among biological pathways, safety constraints, and treatment parameters, thereby reducing computational overhead and increasing optimization speed.
Improving the operation of the computing system by validating the mathematical representation of the patient state against physiological and safety parameters to ensure consistent and reliable generation of health-related recommendations.
Improving the operation of the computing system by updating predictive outputs in real time based on data received from wearable sensors, continuous glucose monitors, and electronic medical records, thereby providing accurate and timely therapeutic guidance in real-time.
Improving the operation of the computing system by enabling closed-loop automation through transmission of actuation signals to an automated drug-delivery device, thereby supporting continuous, adaptive control of insulin therapy and reducing the need for manual intervention.

Exemplary embodiments of the present invention are described largely in the context of a fully functional computer system for encoding an object stream, as is described herein. Readers of skill in the art will recognize, however, that the present invention also may be embodied in a computer program product disposed upon computer readable storage media for use with any suitable data processing system. Such computer readable storage media may be any storage medium for machine-readable information, including magnetic media, optical media, or other suitable media. Examples of such media include magnetic disks in hard drives or diskettes, compact disks for optical drives, magnetic tape, and others as will occur to those of skill in the art. Persons skilled in the art will immediately recognize that any computer system having suitable programming means will be capable of executing the steps of the method of the invention as embodied in a computer program product. Persons skilled in the art will recognize also that, although some of the exemplary embodiments described in this specification are oriented to software installed and executing on computer hardware, nevertheless, alternative embodiments implemented as firmware or as hardware are well within the scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Advantages and features of the present disclosure can be further described by the following statements:

Statement 1. A computer-implemented method for generating one or more health-related recommendations, the computer-implemented method comprising: processing, by a computing device, a plurality of data sources associated with one or more health-related systems to generate integrated pathway parameters; constructing, by the computing device, a mathematical representation of a patient state based on the integrated pathway parameters; dynamically refining, by the computing device, the mathematical representation of the patient state using a machine-learning process configured to adaptively update parameter weights in response to incoming health-related data; and generating, by the computing device and in response to dynamically refining the mathematical representation of the patient state, one or more predictive outputs corresponding to the one or more health-related recommendations.

Statement 2. The computer-implemented method of the statement above, wherein the plurality of data sources comprises at least one of multi-omics data, clinical records, and real-time streaming health data associated with one or more patients.

Statement 3. The computer-implemented method of any combination of one or more of the statements above, wherein constructing the mathematical representation of the patient state further comprises: formulating, by the computing device, a quadratic unconstrained binary optimization model configured to encode one or more relationships among the integrated pathway parameters.

Statement 4. The computer-implemented method of any combination of one or more of the statements above, wherein dynamically refining the mathematical representation of the patient state further comprises: applying, by the computing device, the machine-learning process comprising at least one of a graph neural network, a reinforcement learning model, a neural network prediction model, and a genetic algorithm optimization model.

Statement 5. The computer-implemented method of any combination of one or more of the statements above, wherein generating the one or more predictive outputs further comprises: producing, by the computing device, at least one of an insulin-demand prediction, an insulin-supply optimization, a clinical recommendation, a lifestyle recommendation, and a monitoring plan.

Statement 6. The computer-implemented method of any combination of one or more of the statements above, further comprising: validating, by the computing device, the mathematical representation of the patient state based on one or more predefined health-related constraints, wherein generating the one or more predictive outputs is based on validating the mathematical representation of the patient state.

Statement 7. The computer-implemented method of any combination of one or more of the statements above, further comprising: updating, by the computing device, the one or more predictive outputs based on real-time streaming health data associated with one or more patients received from wearable sensors, glucose monitors, or electronic medical records.

Statement 8. The computer-implemented method of any combination of one or more of the statements above, further comprising: transmitting, by the computing device, an actuation signal to an automated drug-delivery device based on the one or more predictive outputs.

Statement 9. system for generating one or more health-related recommendations, the system comprising: a memory; and a processing device, operatively coupled to the memory, the processing device configured to: process a plurality of data sources associated with one or more health-related systems to generate integrated pathway parameters; construct a mathematical representation of a patient state based on the integrated pathway parameters; dynamically refine the mathematical representation of the patient state using a machine-learning process configured to adaptively update parameter weights in response to incoming health-related data; and generate, in response to dynamically refining the mathematical representation of the patient state, one or more predictive outputs corresponding to the one or more health-related recommendations.

Statement 10. The system of any combination of one or more of the statements above, wherein the plurality of data sources comprises at least one of multi-omics data, clinical records, and real-time streaming health data associated with one or more patients.

Statement 11. The system of any combination of one or more of the statements above, wherein the processing device configured to construct the mathematical representation of the patient is further configured to: formulate a quadratic unconstrained binary optimization model configured to encode one or more relationships among the integrated pathway parameters.

Statement 12. The system of any combination of one or more of the statements above, wherein the processing device configured to dynamically refine the mathematical representation of the patient is further configured to: apply the machine-learning process comprising at least one of a graph neural network, a reinforcement learning model, a neural network prediction model, and a genetic algorithm optimization model.

Statement 13. The system of any combination of one or more of the statements above, wherein the processing device configured to generate the one or more predictive outputs is further configured to: produce at least one of an insulin-demand prediction, an insulin-supply optimization, a clinical recommendation, a lifestyle recommendation, and a monitoring plan.

Statement 14. The system of any combination of one or more of the statements above, wherein the processing device is further configured to: validate the mathematical representation of the patient state based on one or more predefined health-related constraints, wherein generating the one or more predictive outputs is based on validating the mathematical representation of the patient state.

Statement 15. The system of any combination of one or more of the statements above, wherein the processing device is further configured to: update the one or more predictive outputs based on real-time streaming health data associated with one or more patients received from wearable sensors, glucose monitors, or electronic medical records.

Statement 16. The system of any combination of one or more of the statements above, wherein the processing device is further configured to: transmit an actuation signal to an automated drug-delivery device based on the one or more predictive outputs.

Statement 17. A non-transitory computer-readable media storing processor-executable instructions that, when executed by at least one processor, cause the at least one processor to: process a plurality of data sources associated with one or more health-related systems to generate integrated pathway parameters; construct a mathematical representation of a patient state based on the integrated pathway parameters; dynamically refine the mathematical representation of the patient state using a machine-learning process configured to adaptively update parameter weights in response to incoming health-related data; and generate, in response to dynamically refining the mathematical representation of the patient state, one or more predictive outputs corresponding to one or more health-related recommendations.

Statement 18. The computer-readable media of claim 17, wherein the at least one processor is further caused to: validate the mathematical representation of the patient state based on one or more predefined health-related constraints, wherein generating the one or more predictive outputs is based on validating the mathematical representation of the patient state.

Statement 19. The computer-readable media of claim 17, wherein the at least one processor is further caused to: update the one or more predictive outputs based on real-time streaming health data associated with one or more patients received from wearable sensors, glucose monitors, or electronic medical records.

Statement 20. The computer-readable media of claim 17, wherein the at least one processor is further caused to: transmit an actuation signal to an automated drug-delivery device based on the one or more predictive outputs.

It will be understood from the foregoing description that modifications and changes may be made in various embodiments of the present invention without departing from its true spirit. The descriptions in this specification are for purposes of illustration only and are not to be construed in a limiting sense. The scope of the present invention is limited only by the language of the following claims.

What is claimed is:

1. A computer-implemented method for generating one or more health-related recommendations, the computer-implemented method comprising:
processing, by a computing device, a plurality of data sources associated with one or more health-related systems to generate integrated pathway parameters;
constructing, by the computing device, a mathematical representation of a patient state based on the integrated pathway parameters;
dynamically refining, by the computing device, the mathematical representation of the patient state using a machine-learning process configured to adaptively update parameter weights in response to incoming health-related data; and
generating, by the computing device and in response to dynamically refining the mathematical representation of the patient state, one or more predictive outputs corresponding to the one or more health-related recommendations.

2. The computer-implemented method of claim 1, wherein the plurality of data sources comprises at least one of multi-omics data, clinical records, and real-time streaming health data associated with one or more patients.

3. The computer-implemented method of claim 1, wherein constructing the mathematical representation of the patient state further comprises:
formulating, by the computing device, a quadratic unconstrained binary optimization model configured to encode one or more relationships among the integrated pathway parameters.

4. The computer-implemented method of claim 1, wherein dynamically refining the mathematical representation of the patient state further comprises:
applying, by the computing device, the machine-learning process comprising at least one of a graph neural network, a reinforcement learning model, a neural network prediction model, and a genetic algorithm optimization model.

5. The computer-implemented method of claim 1, wherein generating the one or more predictive outputs further comprises:
producing, by the computing device, at least one of an insulin-demand prediction, an insulin-supply optimization, a clinical recommendation, a lifestyle recommendation, and a monitoring plan.

6. The computer-implemented method of claim 1, further comprising:
validating, by the computing device, the mathematical representation of the patient state based on one or more predefined health-related constraints, wherein generating the one or more predictive outputs is based on validating the mathematical representation of the patient state.

7. The computer-implemented method of claim 1, further comprising:
updating, by the computing device, the one or more predictive outputs based on real-time streaming health data associated with one or more patients received from wearable sensors, glucose monitors, or electronic medical records.

8. The computer-implemented method of claim 1, further comprising:
transmitting, by the computing device, an actuation signal to an automated drug-delivery device based on the one or more predictive outputs.

9. A system for generating one or more health-related recommendations, the system comprising:
a memory; and
a processing device, operatively coupled to the memory, the processing device configured to:
process a plurality of data sources associated with one or more health-related systems to generate integrated pathway parameters;
construct a mathematical representation of a patient state based on the integrated pathway parameters;
dynamically refine the mathematical representation of the patient state using a machine-learning process configured to adaptively update parameter weights in response to incoming health-related data; and
generate, in response to dynamically refining the mathematical representation of the patient state, one or more predictive outputs corresponding to the one or more health-related recommendations.

10. The system of claim 9, wherein the plurality of data sources comprises at least one of multi-omics data, clinical records, and real-time streaming health data associated with one or more patients.

11. The system of claim 9, wherein the processing device configured to construct the mathematical representation of the patient is further configured to:
formulate a quadratic unconstrained binary optimization model configured to encode one or more relationships among the integrated pathway parameters.

12. The system of claim 9, wherein the processing device configured to dynamically refine the mathematical representation of the patient is further configured to:
apply the machine-learning process comprising at least one of a graph neural network, a reinforcement learning model, a neural network prediction model, and a genetic algorithm optimization model.

13. The system of claim 9, wherein the processing device configured to generate the one or more predictive outputs is further configured to:
produce at least one of an insulin-demand prediction, an insulin-supply optimization, a clinical recommendation, a lifestyle recommendation, and a monitoring plan.

14. The system of claim 9, wherein the processing device is further configured to:
validate the mathematical representation of the patient state based on one or more predefined health-related constraints, wherein generating the one or more predictive outputs is based on validating the mathematical representation of the patient state.

15. The system of claim 9, wherein the processing device is further configured to:
update the one or more predictive outputs based on real-time streaming health data associated with one or more patients received from wearable sensors, glucose monitors, or electronic medical records.

16. The system of claim 9, wherein the processing device is further configured to:
transmit an actuation signal to an automated drug-delivery device based on the one or more predictive outputs.

17. A non-transitory computer-readable media storing processor-executable instructions that, when executed by at least one processor, cause the at least one processor to:
process a plurality of data sources associated with one or more health-related systems to generate integrated pathway parameters;
construct a mathematical representation of a patient state based on the integrated pathway parameters;
dynamically refine the mathematical representation of the patient state using a machine-learning process configured to adaptively update parameter weights in response to incoming health-related data; and
generate, in response to dynamically refining the mathematical representation of the patient state, one or more predictive outputs corresponding to one or more health-related recommendations.

18. The computer-readable media of claim 17, wherein the at least one processor is further caused to:
validate the mathematical representation of the patient state based on one or more predefined health-related constraints, wherein generating the one or more predictive outputs is based on validating the mathematical representation of the patient state.

19. The computer-readable media of claim 17, wherein the at least one processor is further caused to:
update the one or more predictive outputs based on real-time streaming health data associated with one or more patients received from wearable sensors, glucose monitors, or electronic medical records.

20. The computer-readable media of claim 17, wherein the at least one processor is further caused to:
transmit an actuation signal to an automated drug-delivery device based on the one or more predictive outputs.
